(19) United States
(12) Patent Application Publication
(10) Pub. No.: US 2025/0110136 A1
(43) Pub. Date: Apr. 3, 2025

(54) ASSESSMENT AND DETECTION OF PORTAL VEIN THROMBOSIS IN CHRONIC LIVER DISEASE AND STAGING THEREOF

(71) Applicant: University of Cincinnati, Cincinnati, OH (US)

(72) Inventors: Vladimir Bogdanov, Cincinnai, OH (US); Clayton Lewis, Blue Ash, OH (US); Changchun Xie, Cincinnati, OH (US); Patrick Van Dreden, Guernigou (FR)

(73) Assignee: University of Cincinnati, Cincinnati, OH (US)

(21) Appl. No.: 18/901,605

(22) Filed: Sep. 30, 2024

Related U.S. Application Data
(60) Provisional application No. 63/541,122, filed on Sep. 28, 2023.

(51) Int. Cl. G01N 33/68; G01N 33/84

(52) U.S. Cl. CPC G01N 33/6893; G01N 33/84; G01N 2333/70564; G01N 2333/7454; G01N 2333/7456; G01N 2333/7458; G01N 2333/755; G01N 2333/96461; G01N 2800/085; G01N 2800/56

(57) ABSTRACT

Certain aspects of the disclosure provide systems and methods for diagnosing and treating chronic liver disease and portal vein thrombosis. In particular, methods may include measuring levels of one or more proteins, including factor V, factor VIII, protein C, protein S, D-dimer, sP-selectin, or asTF in a biological sample from a subject and determining a CLD score based on the levels of the one or more proteins, whereby, a CLD stage may be determined based on the CLD score. In some aspects, methods may include determining a PVT score based on levels of the one or more proteins in the biological sample. In some aspects, methods further include treating CLD and/or PVT.

### CROSS-REFERENCE TO RELATED APPLICATIONS

This application claims the benefit of and priority to U.S. Provisional Patent Application No. 63/541,122, filed on Sep. 28, 2023 the entire contents of which are hereby incorporated by reference.

### TECHNICAL FIELD

Aspects of the present disclosure relate to staging chronic liver disease and risk assessment of portal vein thrombosis in chronic liver disease.

### BACKGROUND

Chronic liver disease (CLD) is a progressive deterioration of liver tissue and function. Liver functioning include the production of clotting factors and other proteins, detoxification of harmful products of metabolism, and excretion of bile.

CLD may be due to alcoholic liver disease, non-alcoholic fatty liver disease (NAFLD/NASH), chronic viral hepatitis, or genetic or autoimmune factors. In some rarer cases, CLD may be caused by certain drug use or vascular dysfunction. Alcoholic liver disease includes a spectrum of disease, including alcoholic fatty liver with or without hepatitis, alcohol hepatitis, or cirrhosis. NAFLD/NASH is associated with metabolic syndrome. Chronic viral hepatitis may be due to a hepatitis B, C, or D infection. There are a variety of genetic causes and autoimmune causes of CLD.

Symptoms of CLD may include fatigue, jaundice, abdominal swelling, and bruising.

CLD represents a continuous and progressive process of hepatic fibrosis, liver tissue architectural distortion, and regeneration nodule formation. CLD is the twelfth leading cause of death in the US. Generally, CLD reaches a severity in which the liver transplant is the indicated treatment.

Current methods for quantitative assessment and staging of CLD are based on the Model for End-stage Liver Disease (MELD). MELD assesses the severity of CLD and is used to prioritize patients for liver transplant. MELD is based on bilirubin, international normalized ratio

(INR) and creatinine. Generally, MELD score ranges from 6 to 40, wherein the higher scores are used to indicate greater urgency for transplantation.

MELD-NA is a version of MELD which also incorporates sodium levels as an additional factor. In particular, sodium levels may indicate hyponatremia (low sodium levels), which is a common complication in advanced liver disease and can indicate worse prognosis.

Another scoring system, the Child-Turcotte-Pugh (CTP) system, is configured to assess the severity of CLD and determine prognosis. The CTP system uses total bilirubin, serum albumin, prothrombin time (PT), ascites, and encephalopathy to assess CLD severity. CTP severity is graded into class A, class B, and class C. Class A is for well-compensated liver disease, whereas class B indicates a significant functional compromise. Class C is for decompensated liver disease and often has a poor prognosis.

Prothrombin time (PT) measures clotting time of blood. The measurement may be based on the time in seconds a sample of blood takes to clot or based on a calculation of the international normalized ratio (INR). The INR is a standardized way of expressing the PT result, allowing for consistency across different laboratories. It adjusts for variations in testing methods. An INR of 1.0 is considered normal, while higher values indicate a greater risk of bleeding and are often used to monitor patients on anticoagulant therapy (like warfarin). Elevated PT/INR values may indicate liver dysfunction. INR may be used as a substitute for PT in the CTP method.

Since its introduction in 2002, MELD has helped reduce the mortality rate for those on the liver transplant waiting list by 12%. It is superior at predicting short-term mortality compared to CTP scoring. However, MELD is not perfect, and its modification may further improve outcomes. Prior to 2016, hyponatremia was a risk factor for waitlist mortality. This was solved by incorporation of serum sodium levels in MELD scoring (MELD-Na). However, MELD-Na still has problematic features, e.g., the use of PT/INR, which is a poor indicator of hemostatic balance, and the use of creatinine levels, which directly relate to a patient's muscle mass, and may underrepresent the severity of disease in women and those with poor nutritional status.

Portal vein thrombosis (PVT) is a complication of CLD in which the portal vein becomes narrowed or blocked by a blood clot. The portal vein is the major blood vessel that carries blood from the digestive organs to the liver. PVT occurs in between 4.5% and 25% of CLD patients. Prevalence of PVT increases with the increased CLD severity and increased liver transplant-related morbidity and mortality. This is due to the amount of non-anatomical vascular reconstruction necessary to ensure portal vein patency. Overall survival of liver transplant decreases in patients with PVT. For example, occlusive PVT at the time of liver transplant is associated with an up to seven times increased risk of death 30 days post-operation.

Furthermore, PVT is notoriously difficult to detect due to imaging limitations of ultrasound, CT scan, or MRI. For example, some estimates predict up to 50% of PVT likely remains undetected in CLD patients. Additionally, there are few methods for predicting PVT.

Conventional treatments of PVT include anticoagulation therapies or surgical procedures. Early anticoagulation has a 5-year survival rate of 85%. Additionally, acute PVT has a better prognosis than chronic PVT, where the liver function is often compromised. Surgical options include transjugular intrahepatic portosystemic shunt and open surgery procedures.

As described herein above, there are limitations associated with conventional methods for staging CLD and detection and treatment of PVT. For example, there is currently no model for predicting PVT relying on blood-derived parameters. Accordingly, there is a need for improved methods of staging CLD and assessing PVT.

### SUMMARY

Additional features and advantages of the present disclosure will be set forth in the detailed description, which follows, and in part will be apparent to those skilled in the art from that description or recognized by practicing the aspects described herein, including the detailed description, which follows the claims, as well as the appended drawings.

Certain aspects provide a method of diagnosing and treating a subject having chronic liver disease (CLD), comprising: obtaining a biological sample from the subject; measuring a level of a protein in the biological sample from the subject, wherein the protein comprises one or more of: Factor V, Factor VIII, protein C, protein S, D-dimer, sP-selectin, or asTF; determining a CLD score based on the protein level in the biological sample from the subject; determining a stage of CLD of the subject based on the CLD score; and treating the subject with a CLD treatment based on the stage of CLD.

In certain aspects, the protein level in the biological sample from the subject comprises a plasma concentration of the protein.

In certain aspects, the protein level in the biological sample from the subject comprises an activity level of the protein.

In certain aspects, the protein comprises factor V and factor VIII.

In certain aspects, the protein further comprises one or more of bilirubin or creatinine.

In certain aspects, the method further comprises measuring a sodium level in the biological sample from the subject, and wherein determining the CLD score is further based on the sodium level in the biological sample from the subject.

In certain aspects, determining the stage of CLD of the subject based on the CLD score comprises: comparing the CLD score to a set of threshold values, wherein each threshold value is associated with a CLD stage, and determining the stage of CLD of the subject based on the CLD score satisfying the threshold value of the CLD stage.

In certain aspects, the biological sample from the subject is selected from the group consisting of whole blood, plasma, serum, urine, and saliva.

In certain aspects, a point-of-care (POC) assay is used to measure the protein level of in the biological sample from the subject.

In certain aspects, treating the subject with the CLD treatment based on the stage of CLD comprises transplanting a liver of the subject.

Certain aspects provide a method of diagnosing and treating a subjecting having portal vein thrombosis (PVT) comprising: obtaining a biological sample from the subject; measuring a level of a protein in the biological sample from the subject, wherein the protein comprises one or more of: factor V, factor VIII, protein C, protein S, D-dimer, sP-selectin, or asTF; determining a compensation score based on the level of the protein in the biological sample from the subject; diagnosing the subject as having a PVT based on the compensation score; and administering a PVT treatment to the diagnosed subject.

In certain aspects, the level of the protein in the biological sample from the subject comprises a plasma concentration of the protein.

In certain aspects, the level of the protein in the biological sample from the subject comprises an activity level of the protein.

In certain aspects, the protein comprises one or more of: factor V, factor VIII, or protein S.

In certain aspects, the protein comprises factor V and factor VIII.

In certain aspects, diagnosing the subject as having a PVT based on the compensation score comprises: comparing the compensation score to a PVT threshold value, and determining the PVT based on the compensation score satisfying the PVT threshold value.

In certain aspects, the biological sample from the subject is selected from the group consisting of whole blood, plasma, serum, urine, and saliva.

In certain aspects, a point-of-care (POC) assay is used to measure the level of the protein in the biological sample from the subject.

In certain aspects, administering a PVT treatment to the diagnosed subject comprises: administering an anticoagulation treatment.

In certain aspects, the method further comprises determining a CLD score based on the level of the protein in the biological sample from the subject; determining a stage of CLD of the subject based on the CLD score; and adjusting a compensation score based on the stage of CLD of the subject, wherein diagnosing the subject as having a PVT is based on the adjusted compensation score.

Other aspects provide processing systems configured to perform the aforementioned methods as well as those described herein; non-transitory, computer-readable media comprising instructions that, when executed by a processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

It is to be understood that both the foregoing general description and the following detailed description describe various aspects and are intended to provide an overview or framework for understanding the nature and character of the claimed subject matter. The accompanying drawings are included to provide a further understanding of the various aspects and are incorporated into and constitute a part of this specification. The drawings illustrate the various aspects described herein, and together with the description, explain the principles and operations of the claimed subject matter.

### BRIEF DESCRIPTION OF THE DRAWINGS

The appended figures depict certain aspects and are therefore not to be considered limiting of the scope of this disclosure.

FIG. 1 graphically illustrates box and whisker plots for factor activity (%) or concentration levels of factor V, protein C, sP-Selectin, factor VIII, D-Dimer, and asTF in CLD subjects of a first cohort scored using the Child-Turcotte-Pugh scoring system (CTP-A, CTP-B, or CTP-C). (*p<0.05, **p<0.01, ***p<0.001).

FIG. 2 graphically illustrates box and whisker plots for factor activity (%) or concentration levels of factor V, protein C, sP-Selectin, factor VIII, D-Dimer, and asTF in CLD subjects of a second cohort scored using the Child-Turcotte-Pugh scoring system (CTP-A, CTP-B, or CTP-C). (*p<0.05).

FIGS. 3A-3G depict correlations of factor activity levels and a CLD scoring system with MELD-Na and PT/INR. FIG. 3A depicts correlation of factor V activity levels with MELD-Na. FIG. 3B depicts correlation of protein C activity levels with MELD-Na. FIG. 3C depicts correlation of the CLD scoring system with MELD-Na. FIG. 3D depicts receiver operating characteristic curves for 6-month mortality for MELD-Na scoring system. FIG. 3E depicts receiver operating characteristic curves for 6-month mortality for CLD scoring system. FIG. 3F depicts receiver operating characteristic curves for 1-year mortality for MELD-Na scoring system.

FIG. 3G depicts receiver operating characteristic curves for 1-year mortality for CLD scoring system.

FIGS. 4A-4C depict activity levels of factor VIII, factor V, and protein S, respectively, in patients with CLD awaiting liver transplant. FIG. 4D and FIG. 4E depict factor V and protein S activity levels, respectively, with patient-matched factor VIII activity levels.

FIGS. 5A-5B depict factor V (FIG. 5A) and protein S (FIG. 5B) activity levels with patient matched factor VIII activity levels in CLD patients awaiting liver transplant. Red dots indicate patients with confirmed PVT within 3 months of enrollment. Pink dot indicates patient with confirmed PVT 25 months prior to enrollment.

FIG. 6 depicts an example table comprising factor activity (% 0 and protein concentration levels in PPP) collected from the first cohort previously scored using the Child-Turcotte-Pugh scoring system.

FIG. 7 depicts an example table comprising demographics and clinical characteristics of the second cohort scored using the Child-Turcotte-Pugh scoring system.

FIG. 8 depicts an example table comprising correlation analysis, incidence of PVT versus indicated parameters.

FIG. 9 depicts an example method for diagnosing and treating chronic liver disease.

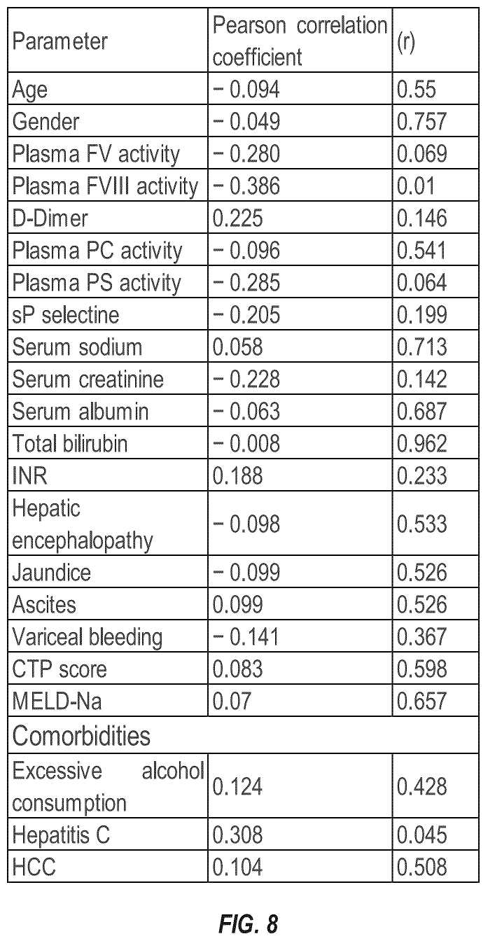

FIG. 8 depicts an example method for diagnosing and treating portal vein thrombosis.

FIG. 11 depicts an example processing system with which aspects of the present disclosure can be performed.

To facilitate understanding, identical reference numerals have been used, where possible, to designate identical elements that are common to the drawings. It is contemplated that elements and features of one aspect may be beneficially incorporated in other aspects without further recitation.

### DETAILED DESCRIPTION

The details of one or more aspects of the presently disclosed subject matter are set forth in this document. Modifications to aspects described in this document, and other aspects, will be evident to those of ordinary skill in the art after a study of the information provided in this document.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some aspects±20%, in some aspects±10%, in some aspects±5%, in some aspects±1%, in some aspects±0.5%, and in some aspects±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

An “effective amount,” as used herein, refers to an amount of a substance (e.g., a therapeutic compound and/or composition) that elicits a desired biological response. In some aspects, an effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a formulation to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective amount may be administered via a single dose or via multiple doses within a treatment regimen. In some aspects, individual doses or compositions are considered to contain an effective amount when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be effective as described herein.

The terms “administer,” “administration,” and “administering,” as used herein, refers to any route of administering an effective amount of a therapeutic agent. In aspects, the administering includes, but is not limited to, intravenous, intraperitoneal, intrapericardial, intramyocardial, intracoronary, subcutaneous, intramuscular, oral, intracerebral, intraspinal, intrathecal, subarachnoid, epidural, periocular, intraocular administration, and the like. In some aspects, the therapeutic agent is administered intramuscularly, subcutaneously, or intravenously.

The terms “treat,” “treatment,” and “treating,” as used herein, refer to a method of alleviating or abrogating a disease, disorder, and/or symptoms thereof in a subject, including a mammal. In certain aspects, the subject is a human patient.

In CLD, there are several coagulation factors known to be altered. These coagulation factors include factor V, which is a cofactor component of the prothrombinase complex. factor V acts a cofactor in the conversion of prothrombin (e.g., factor II) to thrombin (factor Ila). factor V is activated to factor Va by thrombin, or activated factor VII. Activated factor V (factor Va) is inactivated by activated protein C (APC) in conjunction with protein S, thus regulating clot formation. factor V activity is often suppressed in CLD, and may serve as an indicator of liver failure.

Another coagulation factor, factor VIII, however, is drastically increased in CLD. factor VIII is the cofactor component of the intrinsic tenase complex. factor VIII is a cofactor for factor IXa in the activation of factor X to Xa another cofactor component of the prothrombinase complex. Increased factor VIII in severe CLD is due to increased endothelial production and release of von Willebrand factor (vWF), rather than increased synthesis of factor VIII. vWF binds factor VIII and stabilizes circulating factor VIII, thereby increasing its half-life. Expression of factor VIII is correlated with portal hypertension and liver failure. Further, and surprisingly, factor VIII activity levels in subjects with PVT are lower compared to those without PVT.

Two anti-coagulation proteins, protein C and protein S, are diminished in CLD. protein C is a coagulation regular. In its activated form, it helps prevent excessive clotting by inactivating other clotting factors, such as factor V and factor VIII. This action helps to prevent excessive clot formation and promotes the dissolution of clots. Protein S is a co-factor of protein C. protein S enhances activated protein C's ability to inactivate clotting factor V and factor VII to prevent excessive clotting.

Production of protein C and protein S is dependent on vitamin K for the y-carboxylation of multiple N-terminal glutamic acid residues, which is diminished in CLD, and thus, protein C and protein S production is also diminished. Additionally, decreases in circulating levels of protein C and protein S are associated with increased incidence of PVT.

Therefore, as CLD progresses, the ratio of factor VIII to protein C increases. This leads to the accumulation of intrahepatic microthrombi. Further, there is a strong positive correlation between the extent of microthrombosis and the degree of liver fibrosis. Elevated thrombogensis leads to increased fibrinolysis in CLD. For example, some subjects with hyperfibrinolysis, as assessed by high levels of D-dimer and tissue plasminogen activator, have more frequent bleeding and more severe CLD. D-dimer is a fibrin degradation product that is produced when a blood clot dissolves (called fibrinolysis). It is formed from the breakdown of fibrin. Elevated levels of D-dimer may indicate increased clotting activity.

Further, D-dimer levels may be positively correlated with the degree of CLD severity. Additionally, D-dimer levels may be significantly increased in CTP-C subjects, compared to CTP-A and CTP-B, and also D-dimer levels are higher in CTP-C subjects with PVT.

Furthermore, another hallmark of CLD is progressive derangement of the sinusoidal endothelium. Liver injury promotes de-differentiation of sinusoidal endothelial cells to a more capillarized phenotype. Resultantly, the sinusoidal endothelium begins to express P-selectin, which promotes monocyte infiltration.

P-selectin is a cell adhesion molecule that plays a role in inflammatory response and the process of thrombosis. P-selectin is expressed on the surface of activated endothelial cells and platelets. Soluble(s) P-selectin is released by activated endothelial cells and platelets. In some cases, sP-selectin levels may be either elevated and decreased in CLD, depending on the context. Further, in some cases, high sP-selectin levels were shown to have a positive predictive value of the presence of PVT following splenectomy secondary to portal hypertension.

Alternatively spliced tissue factor (asTF) is a naturally occurring isoform of tissue factor (TF) generated via the omission of exon 5 during the processing of TF's primary transcript. In human and mouse, asTF protein features a unique C-terminus that lacks a transmembrane domain, rendering it soluble. asTF protein is able to associate with a subset on integrins on cell surfaces, which can trigger outside-in signaling programs in a variety of cell types. The soluble isoform of asTF may also be released by activated endothelial cells. asTF may be elevated in CLD. Unlike the highly coagulant full-length form of tissue factor, circulating asTF protein is only minimally coagulant and therefore unlikely to be consumed in clotting reactions.

The current gold standard for tracking CLD progression in liver transplant candidates, MELD-Na, assigns a score based on a patient's levels of bilirubin and creatinine (indicators of liver and kidney metabolic health, respectively) as well as PT/INR. Monitoring coagulation is useful as the liver is the main site of synthesis for pro- and anti-hemostatic factors. Expression of these factors is increasingly deranged with worsening disease severity. Given that PT/INR only measures clot formation and not dissolution, the appropriateness of its use to assess coagulative potential in CLD patients is debatable; thus, measuring activity levels of key hemostatic factors may serve as a non-inferior option to the use of PT/INR in MELD-Na scoring.

Aspects of the present disclosure provide for systems and methods for staging and treatment of CLD. In certain aspects, protein levels of proteins associated with CLD are assessed to determine a CLD score. The CLD score may be used to determine a stage of CLD, for example, a first stage, a second stage, and so forth. Further, based on the stage of CLD, one or more treatments may be given to the subject to treat, reduce symptoms, or prevent progression of CLD in the subject.

In certain aspects, one or more protein levels are measured in a biological sample of a subject. Various techniques are suitable for use in measuring the one or more protein levels in the biological sample. In aspects, suitable biological samples include, but are not limited to, whole blood, plasma, serum, urine, and saliva. In a specific aspect, the subject biological sample is whole blood. In another specific aspect, the subject biological sample is plasma.

In aspects, the one or more protein levels are measured in a biological sample of a subject as a point-of-care (POC) assay or method.

In certain aspects, one or more protein levels may be measured using coagulation assays, such as a factor-deficient assay. For example, a factor-deficient assay may be used to measure the functionality of one or more coagulation factors in the biological sample from the subject. In particular, a factor-deficient assay are able to identify specific factor deficiencies or inhibitors. A factor-deficient assay may involve performing a prothrombin time (PT) and an activated partial thromboplastin time (aPTT), except that a reference biological sample known to be deficient in a specific factor type is combined with a subject's biological sample. The resultant time is compared to a standard curve. Thereby, the percent of activity and amount of correction with reference to the standard curve determines the specific factor deficiencies. For example, a factor-deficient assay may measure various factors including factor I, factor II, factor V, factor VII, factor VIII, factor IX, factor X, factor XI, factor XII, factor XIII, protein C, protein S, or thrombomodulin.

As used herein, PT is a measurement of the time it takes for a biological sample, including blood, to clot after adding tissue factor. PT measures coagulation and is reported as a standardized number known as international normalized ratio (INR). During a PT assay, a venous blood sample is obtained and an anticoagulant, such as liquid sodium citrate is added to bind calcium in the sample. The sample is mixed and centrifuged to separate cells from plasma. A plasma sample is extracted and calcium is added to reverse the effects of the anticoagulant, enabling the sample to clot. Then, tissue factor (factor III) is added to activate the extrinsic pathway of coagulation and the time for the sample to clot is measured optically.

As used herein, aPTT is a measurement of the time it takes for a biological sample, including blood, to clot after adding an activator and phospholipids. An aPTT assay is similar to a PT assay in that a venous blood sample is obtained and an anticoagulant is added. The anticoagulant may be oxalate or citrate. The sample is mixed and centrifuged to separate cells from plasma. A plasma sample is extracted and calcium (in a phospholipid suspension) is added. Then, an activator is added to activate the intrinsic pathway of coagulation, for example, silica, celite, kaolin, ellagic acid. The time for the sample to clot is measured optically.

In certain aspects, one or more protein levels may be measured using a quantitative immune-turbidimetric assay. For example, quantitative immune-turbidimetric assay may include a D-dimer assay to quantify the level of D-dimer in a subject's biological sample.

In certain aspects, one or more protein levels may be measured using an Enzyme-Linked Immunosorbent Assay (ELISA). ELISA is a widely used laboratory technique designed to detect and quantify specific proteins, antibodies, hormones, or other molecules in a sample, typically biological fluids such as serum, plasma, or saliva. For example, an ELISA may be used to measure protein levels of sP-selectin or asTF protein.

In aspects, one or more protein levels are quantified via mass spectrometry. Suitable mass spectrometry methods suitable for use in the present methods include, but are not limited to, liquid chromatography-mass spectrometry (LC-MS), ultra-performance liquid chromatography-mass spectrometry (UPLC-MS), triple quadrupole MS, time-of-flight MS, desorption electrospray ionization-mass spectrometry (DESI-MS), and the like.

In certain aspects, a protein level may be a concentration level of a protein in the biological sample from the subject. In certain aspects, a protein level may be an activity level of a protein in the biological sample from the subject.

As described herein, protein levels of some proteins are found to increase with occurrence and progression of CLD, while other proteins are found to decrease with occurrence and progression of CLD. For example, activity levels of factor V, protein C, and protein S may progressively decrease with progression of CLD severity. Activity levels of factor VIII, and thrombomodulin, however, may progressively increase with progression of CLD severity. As another example, circulating levels of D-dimer and asTF protein increase with progression of CLD severity.

In certain aspects, one or more protein levels may be used to determine a CLD score associated with the subject. For example, a CLD score may be determined based on protein levels of one or more of: serum creatinine, serum albumin, total bilirubin, factor V, factor VIII, protein C, protein S, D-dimer, sP-selectin, and asTF. In some aspects, a CLD score may be determined based on an activity level of one or more proteins in a biological sample from the subject, for example, an activity level of factor V, an activity level of factor VIII, and activity level of protein C, or an activity level of protein S. In some aspects, a CLD score may be determined based on a concentration level of one or more proteins, for example, a concentration level of D-dimer, a concentration level of sP-selectin, or a concentration level of asTF.

In some aspects, a CLD score may be additionally, or alternatively, based one or more electrolyte levels associated with the subject. For example, a CLD score may be determined based on a sodium level, such as a sodium concentration in a biological sample from the subject.

In some aspects, a CLD score may be additionally, or alternatively, based on a PT/INR associated with the subject, such as a PT/INR of a biological sample from the subject.

In one aspect, a CLD score may be determined in accordance with Equation 1:

$$CLD Score = 3.78 \times ln ( bilirubin ) + 9.57 \times ln ( creatine ) - 1.16 \times ln ( FV \% ) - 3.1 - ln ( PC \% ) + 29 \qquad ( 1 )$$

Where bilirubin is the concentration level of total bilirubin measured in the biological sample from the subject; creatinine is the concentration level of creatinine measured in the biological sample from the subject; FV % is the activity level of factor V measured in the biological sample from the subject; and PC % is the activity level of protein C measured in the biological sample from the subject. Bilirubin and creatinine may be measured in mg/dL.

In some aspects, a stage of CLD of the subject may be determined based on the CLD score. A stage of CLD of the subject may indicate a severity of CLD for the subject. For example, there may be three stages of CLD, including a first stage, a second stage, and a third stage. A first stage may be for well-compensated CLD and associated with a lower risk of mortality, for example, a mild stage of CLD. A second stage may be for functional compromised CLD and associated with a moderate risk of mortality, for example, a moderate stage of CLD. A third stage may be for decompensated CLD and associated with a higher risk of mortality, for example, a severe stage of CLD. In some aspects, additional or fewer stages of CLD may be utilized.

In some aspects, a stage of CLD of the subject may be determined based on comparing the CLD score of the subject with a set of threshold values. Each threshold value may be associated with a stage of CLD. For example, a threshold value, or a threshold range of values, may be associated with each stage of CLD and the stage of CLD of the subject may be determined by which value(s) the CLD score of the subject satisfies. A threshold value may be determined based on a standard or reference value for CLD stages.

Treatment for CLD may depend on the stage of CLD, indicating the severity of the disease. For example, in advanced liver disease, the treatment may include liver transplant. In some cases, treatment for CLD may depend on the type of CLD, for example, an underlying cause such as viral, autoimmune, toxin, or genetic. In some cases, treatment for CLD may include management of one or more complications and/or symptoms of CLD.

CLD due to chronical viral hepatitis may be treated with antiviral drugs such as continuous viral suppression with nucleoside and nucleotide analogs, direct-acting antivirals achieving HCV eradication, or interferon-alpha. CLD due to alcoholic liver disease may be treated with alcohol abstinence. CLD due to non-alcoholic fatty liver disease may be treated in conjunction with metabolic disorder. CLD due to autoimmune disorder may be treated with corticosteroids and/or other immunosuppressive treatments. CLD due to hereditary hemochromatosis may be treated with phlebotomy and/or iron-chelators. CLD due to Wilson disease may be treated with copper chelators. CLD due to alpha-1-antitrypsin deficiency may be treated by liver transplant. CLD due to primary biliary cholangitis (PBC) may be treated with Ursodeoxycholic acid (UDCA). CLD due to Budd-Chiari syndrome may be treated with anticoagulation, thrombolysis or angioplasty with or without stenting, TIPS, or liver transplant.

In some aspects, a method of diagnosing and treating a subject having chronic liver disease (CLD), comprises obtaining a biological sample from the subject; measuring a level of a protein in the biological sample from the subject, wherein the protein comprises one or more of: factor V, factor VIII, protein C, protein S, D-dimer, sP-selectin, or asTF; determining a CLD score based on the level of the protein in the biological sample from the subject; determining a stage of CLD of the subject based on the CLD score; and treating the subject with a CLD treatment based on the stage of CLD.

In some aspects, the level of the protein in the biological sample from the subject comprises a plasma concentration of the protein. In some aspects, the level of the protein in the biological sample from the subject comprises an activity level of the protein. In some aspects, the protein comprises factor V and factor VIII. In some aspects, the protein further comprises one or more of bilirubin or creatinine.

In some aspects, the method further comprises measuring a sodium level in the biological sample from the subject, and wherein determining the CLD score is further based on a sodium level in the biological sample from the subject.

In some aspects, determining the stage of CLD of the subject based on the CLD score comprises: comparing the CLD score to a set of threshold values, wherein each threshold value is associated with a CLD stage, and determining the stage of CLD of the subject based on the CLD score satisfying the threshold value of the CLD stage.

In some aspects, the biological sample from the subject is selected from the group consisting of whole blood, plasma, serum, urine, and saliva. In some aspects, a point-of-care (POC) assay is used to measure the level of the protein in the biological sample from the subject.

In some aspects, the subject may be treated based on the stage of CLD. For example, in some aspects, in a severe stage of CLD, the treatment may comprise transplanting a liver of a subject.

In some aspects, in a mild stage of CLD, the treatment may include management of metabolic dysfunction. For example, the treatment may include cessation of toxins, such as alcohol. As another example, the treatment may include management of metabolic factors, such as blood sugar and blood lipids. In yet another example, the treatment may include a treatment for metabolic dysfunction.

In some aspects, in a moderate stage of CLD, the treatment may comprise administering one or more medications. For example, a diuretic to reduce fluid volume, a blood pressure reduction medication to reduce portal hypertension, an anti-inflammatory, or anti-viral medication.

In some aspects, a treatment for a subject may further depend on an etiology of the CLD, for example, viral, autoimmune, toxin, or genetic. For example, CLD due to chronical viral hepatitis may be treated with antiviral drugs such as continuous viral suppression with nucleoside and nucleotide analogs, direct-acting antivirals, or interferon-alpha. Meanwhile, CLD due to alcoholic liver disease may be treated with alcohol abstinence. CLD due to non-alcoholic fatty liver disease may be treated in conjunction with metabolic disorder. CLD due to autoimmune disorder may be treated with corticosteroids and/or other immunosuppressive treatments. CLD due to hereditary hemochromatosis may be treated with phlebotomy and/or iron-chelators. CLD due to Wilson disease may be treated with copper chelators. CLD due to alpha-1-antitrypsin deficiency may be treated by liver transplant. CLD due to primary biliary cholangitis (PBC) may be treated with Ursodeoxycholic acid (UDCA). CLD due to Budd-Chiari syndrome may be treated with anticoagulation, thrombolysis or angioplasty with or without stenting, TIPS, or liver transplant.

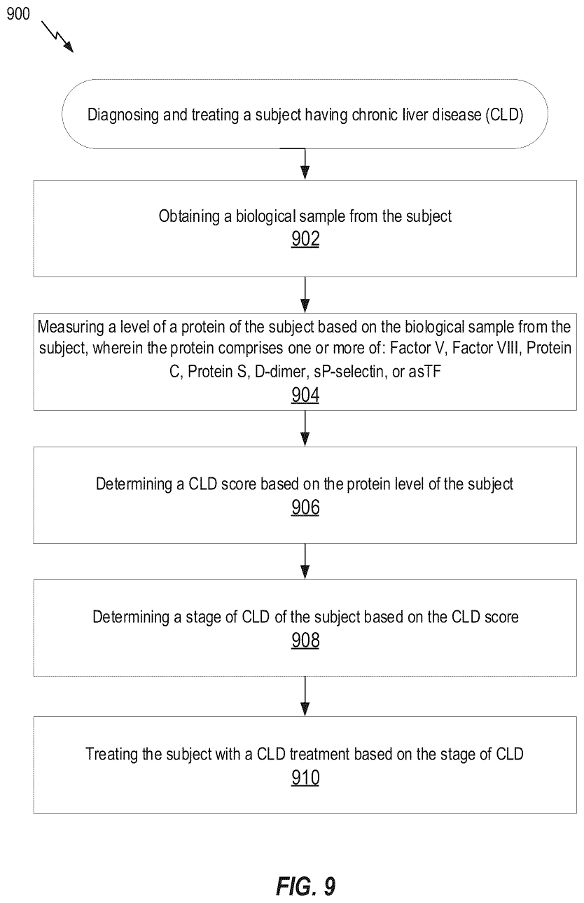

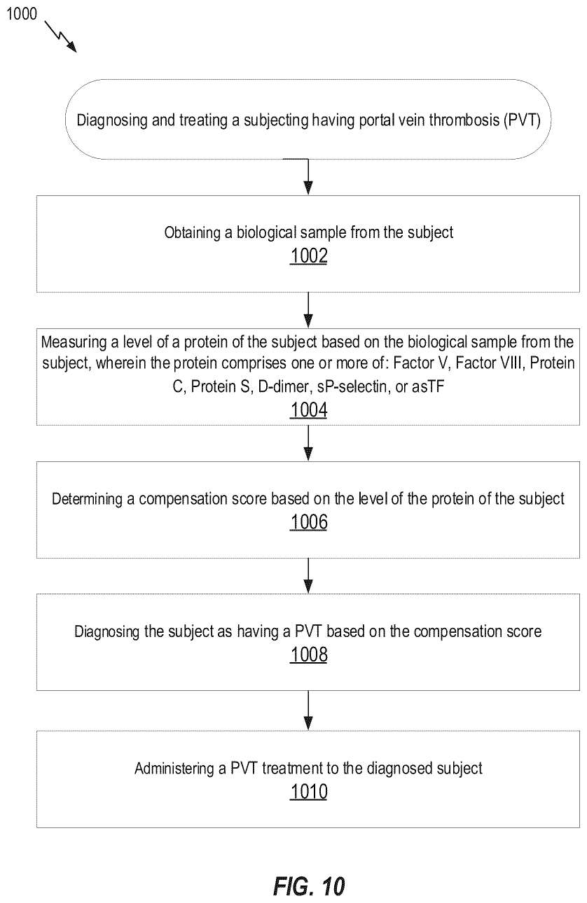

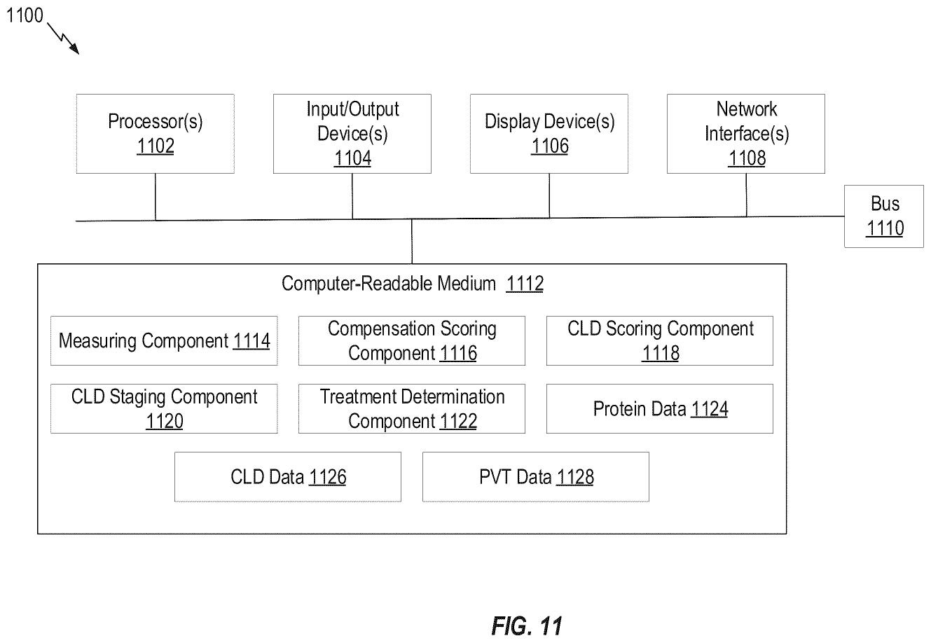

FIG. 9 depict an example method 900 of diagnosing and treating a subject having CLD.

Method 900 begins at step 902 with obtaining a biological sample from the subject. In certain aspects, the biological sample from the subject is selected from the group consisting of whole blood, plasma, serum, urine, and saliva.

Method 900 proceeds to step 904 with measuring a level of a protein of the subject based on the biological sample from the subject, wherein the protein comprises one or more of: factor V, factor VIII, protein C, protein S, D-dimer, sP-selectin, or asTF. In certain aspects, a point-of-care (POC) assay is used to measure the protein level of the subject based on the biological sample from the subject.

In certain aspects, the protein level of the subject comprises a plasma concentration of the protein. In certain aspects, the protein level of the subject comprises an activity level of the protein. In certain aspects, the protein comprises factor V and factor VIII. In certain aspects, the protein further comprises one or more of bilirubin or creatinine.

Method 900 further proceeds to step 906 with determining a CLD score based on the protein level of the subject.

In certain aspects, determining the stage of CLD of the subject based on the CLD score comprises: comparing the CLD score to a set of threshold values, wherein each threshold value is associated with a CLD stage, and determining the stage of CLD of the subject based on the CLD score satisfying the threshold value of the CLD stage.

In certain aspects, the method 900 further comprises measuring a sodium level in the biological sample from the subject, and wherein determining the CLD score is further based on the sodium level in the biological sample from the subject.

Method 900 further proceeds to step 908 with determining a stage of CLD of the subject based on the CLD score.

Method 900 further proceeds to step 910 with treating the subject with a CLD treatment based on the stage of CLD. In certain aspects, treating the subject with the CLD treatment based on the stage of CLD, comprises transplanting a liver of the subject.

Note that FIG. 9 is just one example of a method, and other methods including fewer, additional, or alternative operations are possible consistent with this disclosure.

As described herein, PVT is the narrowing or blockage of the portal vein by a blood clot. PVT often presents with CLD of the liver but can also be associated with various conditions such as malignancy, abdominal sepsis, pancreatitis, systemic lupus erythematosus, and hypercoagulable states. PVT is a common complication of CLD. Occlusive PVT at the time of liver transplant is associated with an up to seven times increased risk of death 30 days post-op.

The portal venous system originates from the vitelline venous system, close to the umbilical venous system, from the fourth to the twelfth weeks of gestation. The portal venous system drains blood from the gastrointestinal track and biliopancreatic apparatus, including the spleen, pancreas, and gallbladder, to the liver. The veins that collect blood from these organs run alongside the major arteries supplying the foregut, midgut, and hindgut, such as the celiac, superior mesenteric, and inferior mesenteric arteries. Ultimately, these veins converge into the portal vein, creating a single venous pathway to the liver. The celiac vein drains structures of the foregut, including the stomach up to the second part of the duodenum. The superior mesenteric vein handles drainage from the third part of the duodenum through the first two-thirds of the transverse colon. The inferior mesenteric vein collects blood from the remaining portion of the transverse colon to the rectum. Together, these veins effectively transport nutrients and toxins from digestion, supplying approximately 75% of the liver's blood flow, while the hepatic artery provides the remaining blood, which then drains into the hepatic veins and returns to the systemic circulation.

PVT can result in complete or partial occlusion of the portal vein and consequent clot propagation into the mesenteric and splenic tributaries. Portal vein occlusion with thrombosis formation typically occurs in two main groups of subjects: (1) subjects with CLD, and (2) subjects with prothrombotic disorders. Acute PVT occurs with abrupt thrombotic portal vein occlusion. Portal hypertension and/or cavernous portal transformation may be features of chronic PVT.

The pathophysiology of PVT encompasses one or more features of the Virchrow triad, which includes reduced portal blood flow, a hypercoagulable state, or vascular endothelial injury. Subjects with CLD usually have slow blood flow through the severely scarred liver. These altered portal hemodynamics are more likely to produce clots and may cause PVT.

The prevalence of PVT in CLD may be between 0.6% to 16% and is more common in subjects awaiting liver transplantation. PVT may be in up to 35% of subjects with CLD and hepatocellular carcinoma. The lifetime risk of PVT in the general population is 1%. factors associated with a higher risk of PVT in subjects with CLD include CPT class B or class C, higher levels of D-dimer, history of non-selective beta-blockers intake, subjects at risk for moderate to severe esophageal varices, and presence of ascites.

Diagnosis of PVT often relies on imaging to determine whether the portal vein is blocked. For example, Doppler ultrasound may be used to show isoechoic or hypoechoic material within the portal vein, partially or entirely filling the lumen with reduced portal venous flow. Ultrasound may also pick up associated with splenomegaly. Contrast-enhanced ultrasound and endoscopic ultrasound are other modalities that are superior to ultrasound in demonstrating the presence or absence of flow in the portal vein when it is very small.

Computed tomography (CT) and magnetic resonance imaging (MRI) may also be used. A CT scan with contrast may also help distinguish a bland thrombus from a malignant one. A bland thrombus is typically seen as a low-density, non-enhancing defect within portal veins. Alternatively, a tumor thrombus enhances following contrast administration with distension of the vessel wall or intra-thrombus contrast enhancement due to neovascularization. MRI is valuable in determining the resectability of neoplasm involving the portal venous system and follow-up after therapeutic procedures. Positron emission tomography CT also has been shown to help differentiate benign and malignant portal vein obstruction.

Aspects of the present disclosure provide for detecting and treating PVT in a subject with PVT. In certain aspects, protein levels of proteins associated with CLD and coagulation of blood are assessed to determine a compensation score for the subject. The compensation score may indicate a risk of developing PVT. Based on the compensation score, one or more treatments may be given to the subject to treat, reduce systems, or prevent development of PVT. For example, a treatment may include a prophylactic treatment of PVT, such as an anticoagulant.

In certain aspects, one or more protein levels are measured in a biological sample of a subject. Various techniques are suitable for use in measuring the one or more protein levels in the biological sample. In aspects, suitable biological samples include, but are not limited to, whole blood, plasma, serum, urine, and saliva. In a specific aspect, the subject biological sample is whole blood. In another specific aspect, the subject biological sample is plasma.

In aspects, the one or more protein levels are measured in a biological sample of a subject as a point-of-care (POC) assay or method. For example, in patients awaiting liver transplant, monitoring the activity of factor VIII along with either factor V and/or protein S may comprise a straightforward, “point-of-care friendly” means to assess short-term risk for PVT.

In certain aspects, one or more protein levels may be measured using coagulation assays, such as a factor-deficient assay. For example, a factor-deficient assay may be used to measure the functionality of one or more coagulation factors in the biological sample from the subject. In particular, a factor-deficient assay are able to identify specific factor deficiencies or inhibitors. A factor-deficient assay may involve performing a PT and an aPTT, except that a reference biological sample known to be deficient in a specific factor type is combined with a subject's biological sample. The resultant time is compared to a standard curve. Thereby, the percent of activity and amount of correction with reference to the standard curve determines the specific factor deficiencies. For example, a factor-deficient assay may measure various factors including factor I, factor II, factor V, factor VII, factor VIII, factor IX, factor X, factor XI, factor XII, factor XIII, protein C, protein S, or thrombomodulin.

In certain aspects, one or more protein levels may be measured using a quantitative immune-turbidimetric assay. For example, quantitative immune-turbidimetric assay may include a D-dimer assay to quantify the level of D-dimer in a subject's biological sample.

In certain aspects, one or more protein levels may be measured using an ELISA. For example, an ELISA may be used to measure protein levels of sP-selectin, or asTF protein.

In aspects, one or more protein levels are quantified via mass spectrometry. Suitable mass spectrometry methods suitable for use in the present methods include, but are not limited to, liquid chromatography-mass spectrometry (LC-MS), ultra-performance liquid chromatography-mass spectrometry (UPLC-MS), triple quadrupole MS, time-of-flight MS, desorption electrospray ionization-mass spectrometry (DESI-MS), and the like.

In certain aspects, a protein level may be a concentration level of a protein in the biological sample from the subject. In certain aspects, a protein level may be an activity level of a protein in the biological sample from the subject.

As described herein, activity of several hemostatic proteins, including, for example, factor V, factor VIII, protein C, and/or protein S may be lower in subjects with or at risk of PVT. However, sP-selectin levels may be higher. For example, in subjects with severe CLD who had factor V activity below 50% and factor VIII activity below 200%, may be correlated with increased incidence of PVT. Thus, factor V and factor VIII activity below these thresholds may comprise a harbinger of hemostatic decompensation and/or cofactor consumption in CLD. As a consequence of hemostatic compensation and increased production of vWF, factor VIII activity levels may be progressively elevated with worsening CLD severity.

However, lower levels of plasma factor VIII activity may correspond with PVT occurrence. For example, high factor VIII activity may be necessary for hemostatic rebalancing in the CLD subject. Whether declining factor VIII activity levels are the cause of, or a result of, consumptive coagulopathy remains to be elucidated. However, it may be that plasma activity levels of factor VIII which would be in the “normal range” for a healthy subject should be viewed with concern in the CLD subject, especially in later disease stages.

In certain aspects, one or more protein levels may be used to determine a compensation score associated with the subject. The compensation score may indicate a risk for a subject developing PVT. For example, a high compensation score may indicate a high risk of developing PVT. A low compensation score may indicate a low risk of developing PVT.

In one aspect, a compensation score may be determined in accordance with Equation 2:

$$CS - 1 = - 6.09 \times ln ( FV ) - 4.66 \times ln ( FVIII \% ) + 48 \qquad ( 2 )$$

Where FV % is the activity level of factor V measured in the biological sample from the subject; and FVIII % is the activity level of factor VIII measured in the biological sample from the subject.

In certain aspects, a subject may be diagnosed with PVT based on the compensation score satisfying a PVT threshold value. For example, a PVT threshold value may be 0, and a subject may be diagnosed with PVT where CS−1 score>0. As another example, a PVT threshold value may be 5.00, and a subject may be diagnosed with PVT where CS−1>5.00.

In one aspect, a compensation score may be determined in accordance with Equation 3:

$$CS - 2 = - 2.27 \times ln ( PS \% ) - 4.34 \times ln ( FVIII \% ) + 50.5 \qquad ( 3 )$$

Where PS % is the activity level of protein S measured in the biological sample from the subject; and FVIII % is the activity level of factor VIII measured in the biological sample from the subject.

In certain aspects, a subject may be diagnosed with PVT based on the compensation score satisfying a PVT threshold value. For example, a PVT threshold value may be 0, and a subject may be diagnosed with PVT where CS−2 score>0. As another example, a PVT threshold value may be 8.00, and a subject may be diagnosed with PVT where CS−2>8.00.

In one aspect, a compensation score may be determined in accordance with Equation 4:

$$Compensation Score ( CS ) = - 67.17 \times ln ( FV \% ) - 3.73 \times ln ( FVIII \% ) + 47 \qquad ( 4 )$$

Where FV % is the activity level of factor V measured in the biological sample from the subject; and FVIII % is the activity level of factor VIII measured in the biological sample from the subject.

In certain aspects, a subject may be diagnosed with PVT based on the compensation score satisfying a PVT threshold value. For example, a PVT threshold value may be 0, and a subject may be diagnosed with PVT where CS>0. As another example, a PVT threshold value may be 4.00, and a subject may be diagnosed with PVT where CS>4.0.

In some aspects, a PVT threshold may be adjusted based on the CLD stage of the subject. For example, based on a stage of CLD, such as determined by a CLD score, a PVT threshold may be adjusted to account for severity of CLD of the subject.

FIG. 10 depicts an example method 1000 of diagnosing and treating a subjecting having portal vein thrombosis (PVT).

Method 1000 begins at step 1002 with obtaining a biological sample from the subject. In some aspects, the biological sample from the subject is selected from the group consisting of whole blood, plasma, serum, urine, and saliva.

Method 1000 proceeds to step 1004 with measuring a level of a protein in a biological sample from the subject, wherein the protein comprises one or more of: factor V, factor VIII, protein C, protein S, D-dimer, sP-selectin, or asTF. In some aspects, the level of the protein in the biological sample from the subject comprises a plasma concentration of the protein. In some aspects, the level of the protein in the biological sample from the subject comprises an activity level of the protein. In some aspects, the protein comprises one or more of: factor V, factor VIII, or protein S. In some aspects, the protein comprises factor V and factor VIII. In some aspects, a point-of-care (POC) assay is used to measure the level of the protein in the biological sample from the subject.

Method 1000 then proceeds to step 1006 with determining a compensation score based on the level of the protein in the biological sample from the subject.

Method 1000 then proceeds to step 1008 with diagnosing the subject as having a PVT based on the compensation score. In some aspects, diagnosing the subject as having a PVT based on the compensation score comprises: comparing the compensation score to a PVT threshold value, and determining the PVT based on the compensation score satisfying the PVT threshold value.

Method 1000 then proceeds to step 1010 with administering a PVT treatment to the diagnosed subject.

In some aspects, administering a PVT treatment to the diagnosed subject comprises administering an anticoagulation treatment. For example, an anticoagulation treatment may be administered to reduce or prevent enlargement of the existing clot in the portal vein. An anticoagulation treatment may, in some examples, also be administered to prevent a further portal vein thrombosis.

In some aspects, administering a PVT treatment to the diagnosed subject comprises administering thrombolytic therapy. Thrombolytic therapy, in some examples, may include a thrombolytic or fibrinolytic agent such as a serine protease. In some cases, a thrombolytic agent may be administered systemically, such as intravenously. In some cases, a thrombolytic agent may be administered locally, such as through a catheter located near the clot.

In some aspects, administering a PVT treatment to the diagnosed subject comprises placement of a transjugular intrahepatic portosystemic shunt (TIPS) with or without thrombectomy. During TIPS, a stent is placed between the portal vein and the hepatic vein, diverting portal venous flow to help reduce portosystemic gradient.

In some aspects, administering a PVT treatment to the diagnosed subject comprises thrombectomy. Thrombectomy is surgical removal of a blood clot. In some cases, a thrombectomy of a clot in the portal vein is performed in conjunction with one or more other PVT treatment.

In some aspects, the method further comprises: determining a CLD score based on the level of the protein in a biological sample from the subject; determining a stage of CLD of the subject based on the CLD score; and adjusting a compensation score based on the stage of CLD of the subject, wherein diagnosing the subject as having a PVT is based on the adjusted compensation score.

Note that FIG. 10 is just one example of a method, and other methods including fewer, additional, or alternative operations are possible consistent with this disclosure.

FIG. 11 depicts an example processing system 1100 configured to perform various aspects described herein, including, for example, method 900 as described above with respect to FIG. 9 or method 1000 as described above with respect to FIG. 10.

Processing system 1100 is generally be an example of an electronic device configured to execute computer-executable instructions, such as those derived from compiled computer code, including without limitation personal computers, tablet computers, servers, smart phones, smart devices, wearable devices, augmented and/or virtual reality devices, and others.

In the depicted example, processing system 1100 includes one or more processors 1102, one or more input/output devices 1104, one or more display devices 1106, one or more network interfaces 1108 through which processing system 1100 is connected to one or more networks (e.g., a local network, an intranet, the Internet, or any other group of processing systems communicatively connected to each other), and computer-readable medium 1112. In the depicted example, the aforementioned components are coupled by a bus 1110, which may generally be configured for data exchange amongst the components. Bus 1110 may be representative of multiple buses, while only one is depicted for simplicity.

Processor(s) 1102 are generally configured to retrieve and execute instructions stored in one or more memories, including local memories like computer-readable medium 1112, as well as remote memories and data stores. Similarly, processor(s) 1102 are configured to store application data residing in local memories like the computer-readable medium 1112, as well as remote memories and data stores. More generally, bus 1110 is configured to transmit programming instructions and application data among the processor(s) 1102, display device(s) 1106, network interface(s) 1108, and/or computer-readable medium 1112. In certain embodiments, processor(s) 1102 are representative of a one or more central processing units (CPUs), graphics processing unit (GPUs), tensor processing unit (TPUs), accelerators, and other processing devices.

Input/output device(s) 1104 may include any device, mechanism, system, interactive display, and/or various other hardware and software components for communicating information between processing system 1100 and a user of processing system 1100. For example, input/output device(s) 1104 may include input hardware, such as a keyboard, touch screen, button, microphone, speaker, and/or other device for receiving inputs from the user and sending outputs to the user.

Display device(s) 1106 may generally include any sort of device configured to display data, information, graphics, user interface elements, and the like to a user. For example, display device(s) 1106 may include internal and external displays such as an internal display of a tablet computer or an external display for a server computer or a projector. Display device(s) 1106 may further include displays for devices, such as augmented, virtual, and/or extended reality devices. In various embodiments, display device(s) 1106 may be configured to display a graphical user interface.

Network interface(s) 1108 provide processing system 1100 with access to external networks and thereby to external processing systems. Network interface(s) 1108 can generally be any hardware and/or software capable of transmitting and/or receiving data via a wired or wireless network connection. Accordingly, network interface(s) 1108 can include a communication transceiver for sending and/or receiving any wired and/or wireless communication.

Computer-readable medium 1112 may be a volatile memory, such as a random access memory (RAM), or a nonvolatile memory, such as nonvolatile random access memory (NVRAM), or the like. In this example, computer-readable medium 1112 includes measuring component 1114, compensation scoring component 1116, CLD scoring component 1118, CLD staging component 1120, treatment determination component 1122, protein data 1124, CLD data 1126, and PVT data 1128.

In certain embodiments, measuring component 1114 is configured to obtain and interpret one or more protein levels measured in a biological sample. In some examples, measuring component 1114 is configured to store the one or more protein levels as protein data 1124.

In certain embodiments, compensation scoring component 1116 is configured to determine a compensation score based on protein data 1124. In some embodiments, compensation scoring component 1116 is configured to determine a compensation score in accordance with one or more of equation 2, 3, and/or 4. In some embodiments, compensation scoring component 1116 is configured to store a compensation score as PVT data 1128.

In certain embodiments, CLD scoring component 1118 is configured to determine a CLD score based on protein data 1124. In some embodiments, CLD scoring component 1118 is configured to determine a CLD score in accordance with equation 1. In some embodiments, CLD scoring component 1118 is configured to store a CLD score as CLD data 1126.

In certain embodiments, CLD staging component 1120 is configured to determine a CLD stage based on the CLD score determined by CLD scoring component 1118. In some embodiments, CLD staging component 1120 is configured to store a CLD stage as CLD data 1126.

Note that FIG. 11 is just one example of a processing system consistent with aspects described herein, and other processing systems having additional, alternative, or fewer components are possible consistent with this disclosure.

### EXAMPLES

The following Examples are offered by way of illustration and are presented in a manner such that one skilled in the art should recognize are not meant to be limiting to the present disclosure as a whole or to the appended claims.

### Example 1: CLD Scoring System

The present inventors investigated plasma activity levels of factor V, factor VIII, protein C, and protein S (protein S) and the concentrations of D-dimer, sP-selectin, and asTF in two cohorts of CLD patients (ambulatory, n=42; liver transplant, n=43).

Two cohorts of subjects were investigated. In Cohort I, whole blood was collected via venipuncture (0.129 mol/L sodium citrate) from healthy subjects (n=30) and CLD patients suffering from cirrhosis due to untreated hepatitis C infection (HCV) and/or excessive alcohol consumption that were previously assigned a CTP score: CTP-A, n=12; CTP-B, n=19; CTP-C, n=11. In Cohort II, blood was collected via venipuncture (0.129 mol/L sodium citrate) from CLD patients awaiting liver transplant. CTP and MELD-Na scores were assessed at the time of blood collection. Cohort II comprised patients suffering from alcoholic and non-alcoholic steatohepatitis, with 9/43 experiencing an HCV comorbidity. The cohort consisted of CTP-A, n=12; CTP-B, n=28; CTP-C, n=3.

Within 2 h post-collection, specimens were centrifuged for 15 min at 1500×g, and then 13,000×g for 2 min to obtain platelet poor plasma (PPP). A sample of the PPP was immediately assessed for factor V activity using Stago STA-Deficient assay kit (Diagnostica Stago) to measure the activity levels of factor V, factor VIII, protein C, protein S, and thrombomodulin (TM). D-dimer levels were measured using STA-liatest D-Di (Diagnostica Stago). The levels of sP-selectin protein were measured in PPP via commercially available ELISA (Millipore Sigma RAB0426). The levels of asTF protein were measured in PPP via custom ELISA as previously described [Unruh D, Sagin F, Adam M, Van Dreden P, Woodhams B J, Hart K, Lindsell C J, Ahmad S A, Bogdanov V Y. Levels of Alternatively Spliced Tissue factor in the Plasma of Patients with Pancreatic Cancer May Help Predict Aggressive Tumor Phenotype. Ann Surg Oncol. 2015; 22:1206-11. DOI: 10.1245/s10434-015-4592-2].

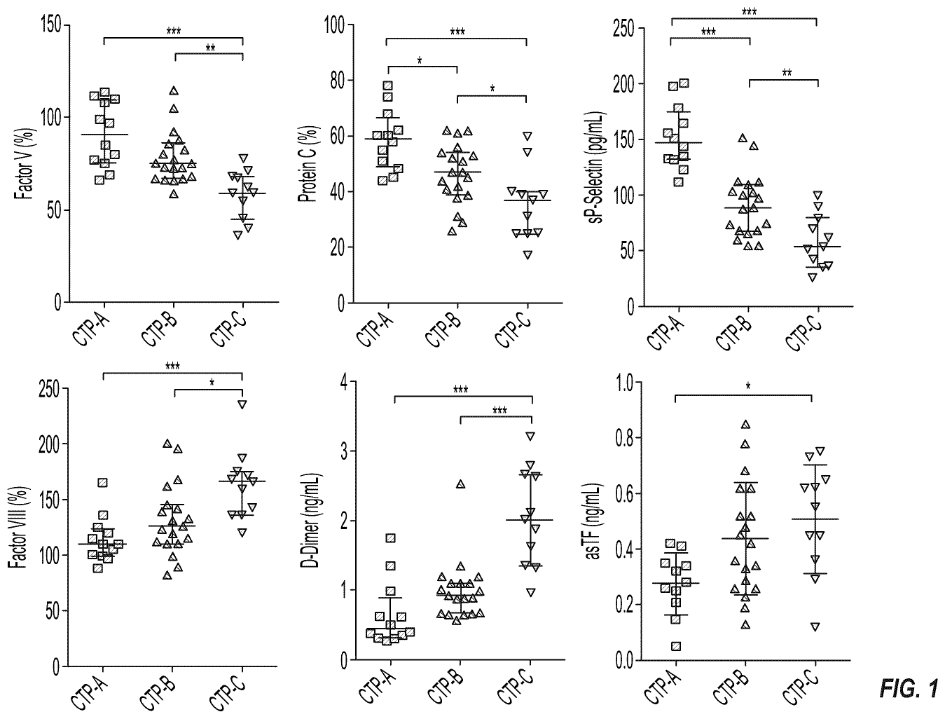

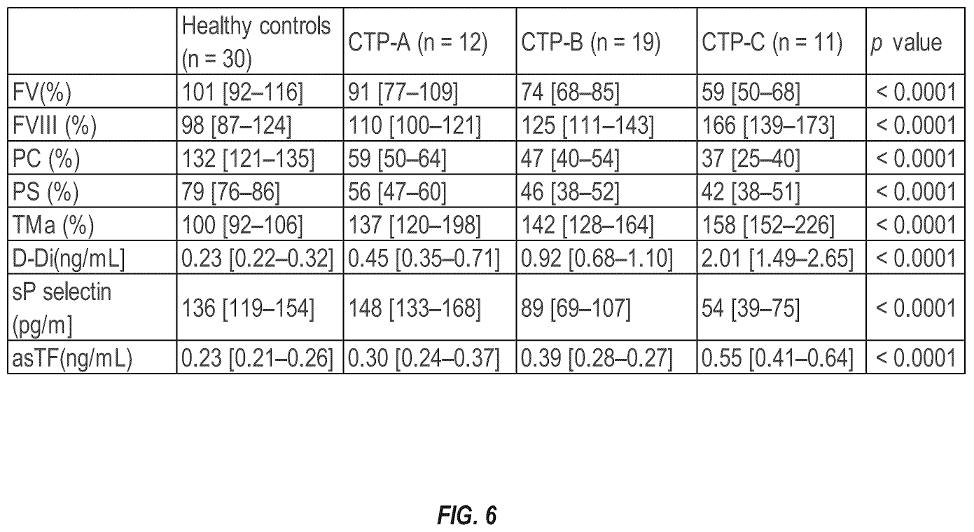

Assessment of PPP specimens of Cohort I showed factor V activity progressively decreased along the spectrum of CLD severity (FIG. 1). protein C and protein S activity levels also decreased with CLD severity (FIG. 6). factor VIII and TM activity progressively increased with disease severity (FIG. 1, FIG. 6). The circulating levels of D-dimer along with those of asTF protein increased with disease severity, while the levels of sP-selectin decreased (FIG. 1).

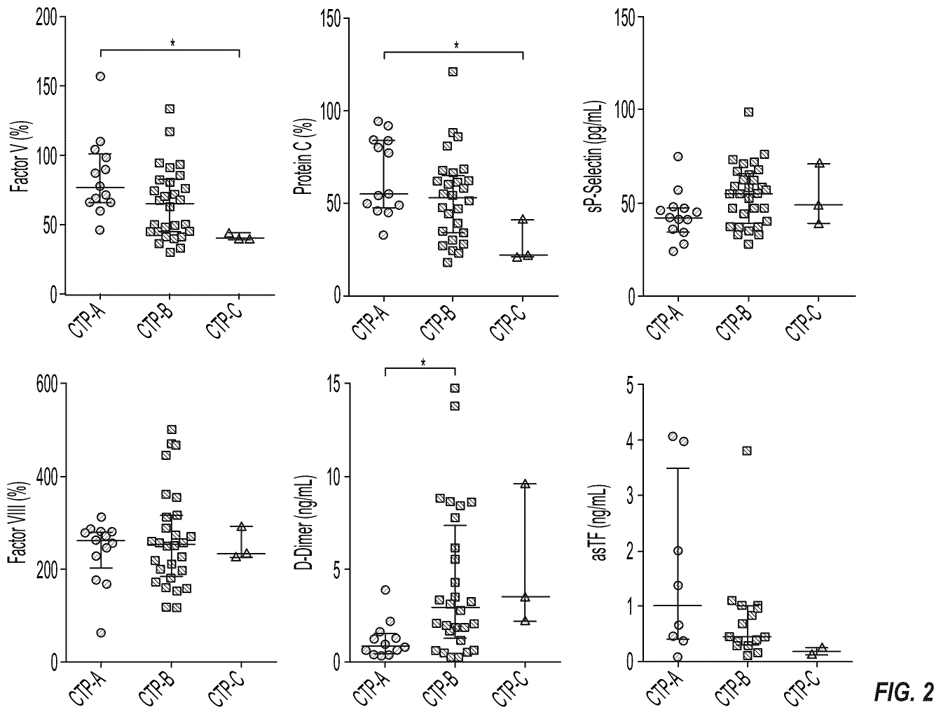

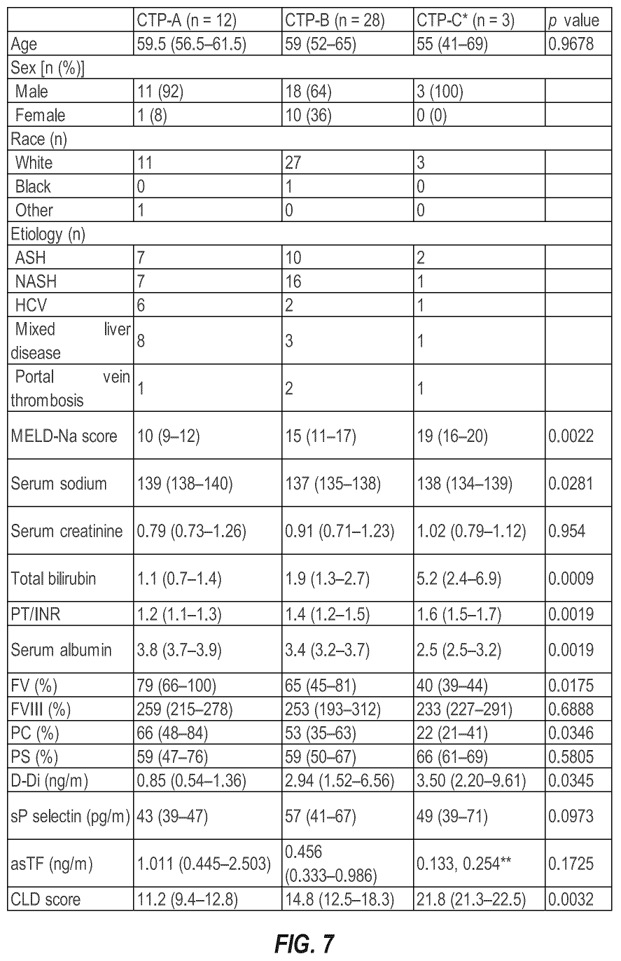

PPP specimens of Cohort II were evaluated for the activity levels of factor V, factor VIII, protein C, protein S, and the levels of D-dimer, sP-selectin, and asTF along with standard CLD evaluation labs: serum sodium, albumin, creatinine, total bilirubin, and PT/INR (FIG. 7). As in Cohort I, factor V and protein C activity declined significantly with CLD progression, while the levels of D-dimer rose (FIG. 2). Also in agreement with the Cohort I were the low levels of protein S activity in all patients (FIG. 7). factor VIII activity was markedly elevated across the entire Cohort II, while sP-selectin protein levels were universally low (FIG. 2). The levels of asTF protein in the majority of subjects were higher than those in healthy individuals (FIG. 2).

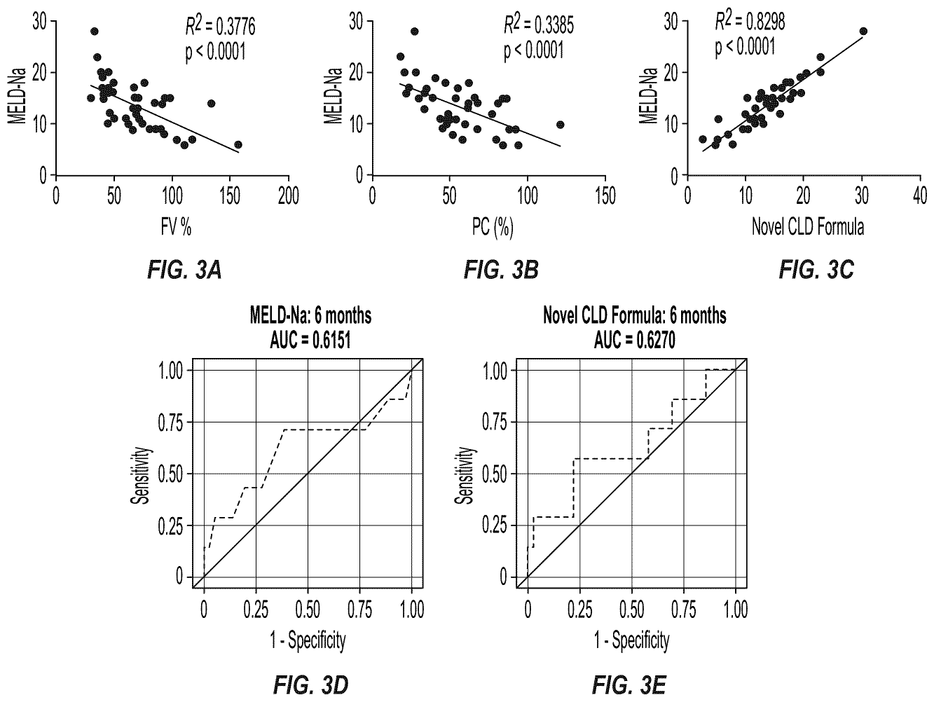

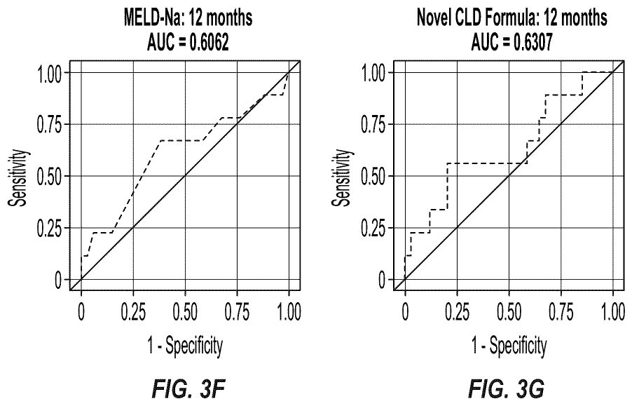

One-way ANOVA analysis was performed using GraphPad Prism (version 6.0; GraphPad Software, San Diego, CA) and used to detect the differences between groups in FIG. 1 and FIG. 2, with p of 0.05 being considered significant. Linear regressions between factor activity levels or plasma concentrations and MELD-Na were performed using GraphPad Prism 6.0. Correlation analyses of factor activity levels in liver transplant subjects and their MELD-Na scores revealed that factor V activity levels significantly correlated with MELD-Na values; interestingly, activity levels of anti-coagulant protein C behaved analogously to those of factor V levels (FIGS. 3A, 3B). Seeing that activity levels of both a pro-coagulant (factor V) and an anti-coagulant (protein C) protein strongly correlated with MELD, factor V and protein C activity levels were next mathematically combined. Multiple linear regression with MELD-Na−3.78×ln(bilirubin)−9.57×ln(creatinine) as dependent variable and ln(factor V %) and ln(protein C %) as independent variables were used to derive the coefficients for ln(factor V %) and ln(protein C %) along with the intercept yielding the CLD scoring system, e.g., Equation 1. All analyses were performed using SAS Version 9.4 (SAS Institute, Cary, NC).

Assessment of this scoring system compared to MELD-Na showed a strongly significant positive correlation (FIG. 3C). At the time of 6-month follow-up, 36 of the 43 liver transplant patients were alive. The seven deceased patients had an average MELD-Na of 15.2+6.4 and an average novel CLD score of 16.2+6.9. Using the c-statistic with 6-month mortality as the endpoint, the area under the receiver operating characteristic (ROC) curves for MELD-Na and this scoring system did not differ significantly. The areas under the curves were 0.615 and 0.627, respectively (FIGS. 3D, 3E). At the time of 1-year follow-up, 33 of the 43 liver transplant patients were living. Using c-statistic with 12-month mortality as the endpoint, the AUC under the ROC were 0.606 and 0.631 for MELD-Na and this scoring system, respectively (FIGS. 3F, 3G). Thus, substituting PT/INR with the activity levels of factor V and protein C provided a non-inferior method to predict 6-month and 1-year mortality compared to MELD-Na. A panel of routinely performed point-of-care plasma assays may aid in CLD management. This study demonstrates that the activity levels of factor V and protein C may hold utility as an alternative means of tracking the hemostatic status in liver transplant candidates.

This is likely attributable to the central nature of these two proteins in the coagulation cascade and how their circulating levels are reflective of hemostatic rebalancing in CLD. Activated factor V (factor Va) is an essential component of prothrombinase, and active protein C regulates factor Va by proteolytic cleavage; as such, factor V and protein C activity levels comprise key indicators of hemostatic capacity. As shown in FIGS. 1 and 2, deficiency in these factors strongly correlates with CLD severity in the ambulatory cohort I, and in the liver transplant cohort II. While maintaining the speed of access to clinical laboratory results, factor V and protein C activity quantification gains advantage over PT/INR due to the greater dynamic range of factor quantification measurements over clotting time measurements, which are typically accurate to only a tenth of a second. Monitoring the activity of both a coagulant and an anti-coagulant plasma factor is likely qualitatively superior to measuring the duration of an artificially induced blood clotting process.

### Example 2: PVT Assessment System

Further, the identification of use of factor V and active protein C as indicators of hemostatic capacity led to the investigation of use of coagulant and anti-coagulant plasma factors to assess short(er)-term risk for PVT in liver transplant.

Two logistic regression analyses were performed on the PPP specimens prepared in Example 1 to develop a compensation score for the prediction of PVT. For the first logistic regression analysis, PVT incidence was dependent, ln(factor V %) and ln(factor VIII %) were independent variables; for the second logistic regression analysis, PVT incidence was dependent, ln(protein S %) and ln(factor VIII %) were independent variables. Their coefficients were used to create the two compensation scores, e.g., Equation 2 and Equation 3.

A significant inverse correlation between factor VIII activity levels and PVT was found in the liver transplant cohort (p=0.010); factor V and protein S activity levels were in-trend (p=0.069, p=0.064). A logistic regression-based compensation score was developed to identify patients at risk of PVT, e.g., Equation 4.

In cohort II of 43 liver transplant patients, there were 3 instances of PVT diagnosed by imaging within 3 months of consent (7%). Patients with diagnosed PVT were followed up clinically via several visits to our liver transplant clinic post-diagnosis; CBC counts were recorded and there were also biochemical and imaging follow-ups comprising updated MELD and CT/MRI/Doppler ultrasound, respectively. Post-hoc correlation analysis between PVT incidence and the measured parameters revealed a strongly significant inverse correlation with factor VIII activity levels (p=0.010; FIG. 8). Activity levels of two other factors, factor V and protein S, also displayed in-trend inverse correlations with PVT incidence (p=0.069 and p=0.064, respectively; FIG. 8).

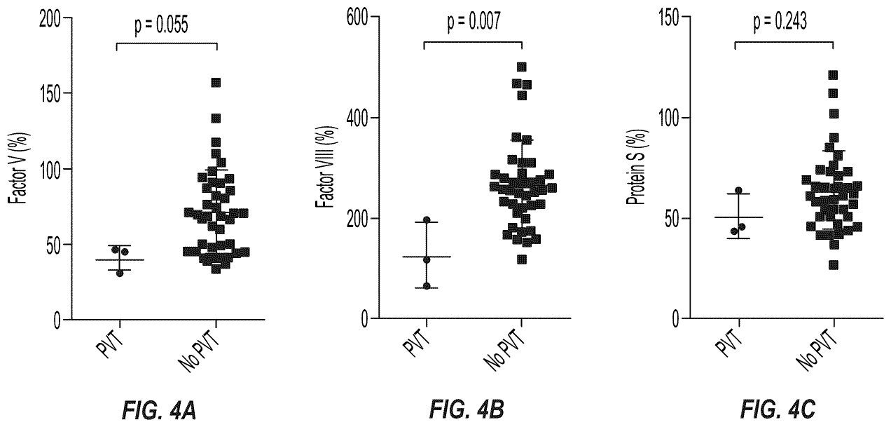

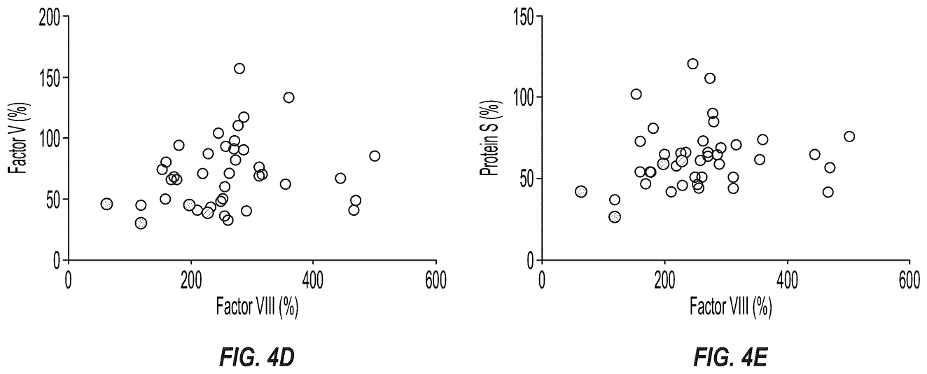

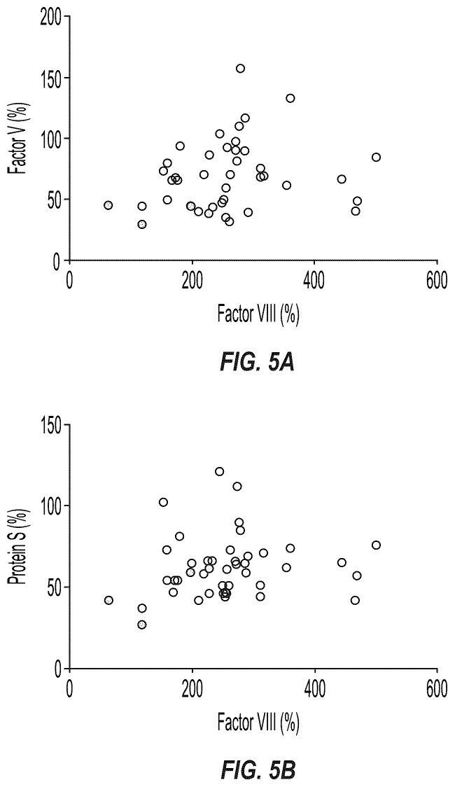

Next, the present inventors performed Mann-Whitney tests to compare the activity levels of factor VIII, factor V, and protein S for patients with and without PVT (FIG. 4A-C). Again, factor VIII levels were found to be significantly lower in patients with PVT, while factor V levels remained in-trend; however, protein S levels, while lower in patients with PVT, were not statistically significantly different between the groups. Given the strength of the correlation between PVT incidence and factor VIII activity, the present inventors matched and graphed each patient's factor V activity levels with the corresponding factor VIII activity levels; factor VIII levels vs protein S levels were also graphed, given the in-trend value obtained for protein S in the correlation analysis (FIGS. 4D, 4E). When distances from the graph origin were calculated for factor V: factor VIII, the three patients with PVT were 1st, 2nd, and 10th closest to the origin (FIG. 4D); in the graph of protein S: factor VIII they were 1st, 2nd, and 11th closest to the origin (FIG. 4E). Logistic regression analysis was carried out to use the activity levels of factor VIII, along with those of factor V and protein S, to create two compensation score equations, CS−1 and CS−2, e.g., Equation 2 and Equation 3.

A total of 11 patients, including the 3 cases of confirmed PVT, had a CS−1 score>0 while a total of 20 patients had a CS−2 score>0. None of the patients with a CS−1 or CS−2 less than 0 were diagnosed with PVT. 2 of the 3 patients with a confirmed PVT had CS−1>5.00 and CS−2>8.00: one had a CS−1 of 5.06, a CS−2 of 8.20 (factor V 30%; factor VIII 118%; CTP-B; MELD-Na 15), was not on anticoagulation at the time of sample collection, and was diagnosed with PVT one day after enrollment while the other had a CS−1 of 5.38, CS−2 of 8.43 (factor V 46%; factor VIII 63%; CTP-A; MELD-Na 12), was diagnosed with PVT 3 months prior to enrollment, and was not on anticoagulation at the time of sample collection. The third patient with a confirmed PVT was not on anticoagulation at the time of sample collection, had a CS−1 of 0.19, a CS−2 of 1.05 (factor V % 45; factor VIII 197%; CTP-B; MELD-Na 17), and was diagnosed with PVT two weeks after sample collection. Thus, multi-factor consumption may be a possible unifying condition among the three patients with PVT.

In cohort II of 43 liver transplant patients, there were 4 instances of PVT diagnosed by imaging. Post-hoc correlation analysis between PVT incidence and the measured parameters revealed a strong inverse correlation with only factor V and factor VIII activity levels (p=0.030 and p=0.016, respectively). When activity levels for these two parameters are matched and graphed, the 4 patients with PVT cluster in the bottom left corner. This a third compensation score (CS) equation by performing a logistic regression, resulting in the Equation 4.

Two patients had a CS>4.0, both of which had a diagnosed PVT. An additional seven patients had a CS between 0 and 4, two of which had a diagnosed PVT. None of the patients with a CS less than 0 were diagnosed with PVT. Of the two patients with CS>4.0, one had a CS of 4.8 (factor V 305; factor VIII 118%), was not on anticoagulation at the time of sample collection, and was diagnosed with PVT one day after enrollment; the other patient had a CS of 4.1 (factor V 46%; factor VIII 63%), was diagnosed with PVT 3 months prior to enrollment, and was not on anticoagulation at the time of sample collection. Of the two patients with CS between 0 and 4, one patient was diagnosed with PVT 25 months prior to enrollment, had a CS of 0.5 (factor V 39%, factor VIII 227%), and was not on anticoagulation at the time of sample collection; the other patient was not on anticoagulation at the time of sample collection, had a CS of 0 (factor V 45%; factor VIII 197%), and was diagnosed with PVT two weeks after sample collection. Thus, consumptive coagulopathy is a possible unifying condition among the four patients with PVT.

Accordingly, monitoring activity of factor VIII along with either factor V and/or protein S may comprise a more straightforward means to assess short-term risk for PVT. It was observed that in patients with severe CLD who had factor V activity below 50% and factor VIII activity below 200%, which corresponded to a CS>0, the incidence of PVT rose drastically. In fact, of the 6 patients who had factor V and factor VIII activity levels less than 50% and 200%, respectively, 3 had a confirmed PVT. Thus, factor V and factor VIII activity below these thresholds may comprise a harbinger of hemostatic decompensation and/or cofactor consumption in CLD. As a consequence of hemostatic compensation and increased production of VWF, factor VIII activity levels are progressively elevated with worsening CLD severity.

### Additional Considerations

The preceding description is provided to enable any person skilled in the art to practice the various aspects described herein. The examples discussed herein are not limiting of the scope, applicability, or aspects set forth in the claims. Various modifications to these aspects will be readily apparent to those skilled in the art, and the generic principles defined herein may be applied to other aspects. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements, and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some” refers to one or more.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the aspects shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method of diagnosing and treating a subject having chronic liver disease (CLD), comprising:
obtaining a biological sample from the subject;
measuring a level of a protein in the biological sample from the subject, wherein the protein comprises one or more of: factor V, factor VIII, protein C, protein S, D-dimer, sP-selectin, or asTF;
determining a CLD score based on the level of the protein in the biological sample from the subject;
determining a stage of CLD of the subject based on the CLD score; and
treating the subject with a CLD treatment based on the stage of CLD.

2. The method of claim 1, wherein the level of the protein in the biological sample from the subject comprises a plasma concentration of the protein.

3. The method of claim 1, wherein the level of the protein in the biological sample from the subject comprises an activity level of the protein.

4. The method of claim 1, wherein the protein comprises factor V and factor VIII.

5. The method of claim 1, wherein the protein further comprises one or more of bilirubin or creatinine.

6. The method of claim 1, further comprising measuring a sodium level in the biological sample from the subject, and wherein determining the CLD score is further based on the sodium level in the biological sample from the subject.

7. The method of claim 1, wherein determining the stage of CLD of the subject based on the CLD score, comprises:
comparing the CLD score to a set of threshold values, wherein each threshold value is associated with a CLD stage, and
determining the stage of CLD of the subject based on the CLD score satisfying the threshold value of the CLD stage.

8. The method of claim 1, wherein the biological sample from the subject is selected from the group consisting of whole blood, plasma, serum, urine, and saliva.

9. The method of claim 1, wherein a point-of-care (POC) assay is used to measure the level of the protein in the biological sample from the subject.

10. The method of claim 1, wherein treating the subject with the CLD treatment based on the stage of CLD comprises transplanting a liver of the subject.

11. A method of diagnosing and treating a subject having portal vein thrombosis (PVT) comprising:
obtaining a biological sample from the subject;
measuring a level of a protein in the biological sample from the subject, wherein the protein comprises one or more of: factor V, factor VIII, protein C, protein S, D-dimer, sP-selectin, or asTF;
determining a compensation score based on the level of the protein in the biological sample from the subject;
diagnosing the subject as having a PVT based on the compensation score; and
administering a PVT treatment to the diagnosed subject.

12. The method of claim 11, wherein the level of the protein in the biological sample from the subject comprises a plasma concentration of the protein.

13. The method of claim 11, wherein the level of the protein in the biological sample from the subject comprises an activity level of the protein.

14. The method of claim 11, wherein the protein comprises one or more of: factor V, factor VIII, or protein S.

15. The method of claim 11, wherein the protein comprises factor V and factor VIII.

16. The method of claim 11, wherein diagnosing the subject as having a PVT based on the compensation score, comprises:
comparing the compensation score to a PVT threshold value, and
determining the PVT based on the compensation score satisfying the PVT threshold value.

17. The method of claim 11, wherein the biological sample from the subject is selected from the group consisting of whole blood, plasma, serum, urine, and saliva.

18. The method of claim 11, wherein a point-of-care (POC) assay is used to measure the level of the protein in the biological sample from the subject.

19. The method of claim 11, wherein administering a PVT treatment to the diagnosed subject comprises administering an anticoagulation treatment.

20. The method of claim 11, further comprising:
determining a CLD score based on the level of the protein of the subject;
determining a stage of CLD of the subject based on the CLD score; and
adjusting the compensation score based on the stage of CLD of the subject, wherein diagnosing the subject as having a PVT is based on the adjusted compensation score.
